SEQUENCE LISTING (38 sequences)

SEQ ID NO: 1 - protein, 25 aa, synthetic construct
  REGION 1..25: note = signal peptide (TPA)
MDAMLRGLCCVLLLCGAVFVSPSHA

SEQ ID NO: 2 - protein, 208 aa, synthetic construct [sequence omitted]

SEQ ID NO: 3 - protein, 30 aa, synthetic construct
  REGION 1..30: note = hinge with linker
GSGGGGSGGGGSGGGGSAESKYGPPCPPCP

SEQ ID NO: 4 - protein, 216 aa, synthetic construct
  REGION 1..216: note = immunoglobulin fc
APEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 5 - protein, 5 aa, synthetic construct
  REGION 1..5: note = linker
GGGGS

SEQ ID NO: 6 - protein, 133 aa, synthetic construct
  REGION 1..133: note = hIL-2M
APTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 617 aa, synthetic construct
  REGION 1..617: note = fusion protein comprising variants of IL-2 and fragments of CD80
MDAMLRGLCCVLLLCGAVFVSPSHAVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - DNA, 1857 bp, synthetic construct
  misc_feature 1..1857: note = nucleotiedes coding fusion protein (GI101)
  source 1..1857: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacctgatga

SEQ ID NO: 9 - protein, 592 aa, synthetic construct
  REGION 1..592: note = fusion protein (GI101)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 133 aa, synthetic construct [sequence omitted]

SEQ ID NO: 11 - protein, 288 aa, synthetic construct
  REGION 1..288: note = CD80
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 12 - protein, 215 aa, synthetic construct
  REGION 1..215: note = modified Fc
SHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 306 aa, synthetic construct
  REGION 1..306: note = mCD80
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 14 - DNA, 1848 bp, synthetic construct
  misc_feature 1..1848: note = nucleotiedes coding fusion protein (mGI101)
  source 1..1848: mol_type = other DNA
atggatgctatgctgagaggcctgtgttgcgtgctgctgctgtgtggcgctgtgttcgtgtctccttctcacgctgtggacgagcagctctccaagtccgtgaaggataaggtcctgctgccttgccggtacaactctcctcacgaggacgagtctgaggaccggatctactggcagaaacacgacaaggtggtgctgtccgtgatcgccggaaagctgaaagtgtggcctgagtacaagaacaggaccctgtacgacaacaccacctacagcctgatcatcctgggcctcgtgctgagcgatagaggcacctattcttgcgtggtgcagaagaaagagcggggcacctacgaagtgaagcacctggctctggtcaagctgtccatcaaggccgacttcagcacccctaacatcaccgagtctggcaacccttccgccgacaccaagagaatcacctgtttcgcctctggcggcttccctaagcctcggttctcttggctggaaaacggcagagagctgcccggcatcaataccaccatttctcaggacccagagtccgagctgtacaccatctccagccagctcgactttaacaccaccagaaaccacaccatcaagtgcctgattaagtacggcgacgcccacgtgtccgaggactttacttgggagaaacctcctgaggaccctcctgactctggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctatagagtggtgtccgtgctgaccgtgctgcaccaggattggctgaacggcaaagagtacaagtgcaaggtgtccaacaagggcctgccttccagcatcgaaaagaccatcagcaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggctctgcacaaccactacacccagaagtccctgtctctgtctcttggaggtggtggcggttctgcccctacctccagctctaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacttgcagtgcctggaagaggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagagcatcatctccacactgacc

SEQ ID NO: 15 - protein, 616 aa, synthetic construct
  REGION 1..616: note = fusion protein (mGI101)
MDAMLRGLCCVLLLCGAVFVSPSHAVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 16 - DNA, 1437 bp, synthetic construct
  misc_feature 1..1437: note = nucleotiedes coding fusion protein (GI101C1)
  source 1..1437: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggccctgcacaatcactacacccagaagtccctgtctctgtccctgggc

SEQ ID NO: 17 - protein, 454 aa, synthetic construct
  REGION 1..454: note = fusion protein (GI101C1)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 18 - DNA, 1176 bp, synthetic construct
  misc_feature 1..1176: note = nucleotiedes coding fusion protein (GI101C2)
  source 1..1176: mol_type = other DNA
atggatgctatgctgagaggcctgtgttgcgtgctgctgctgtgtggcgctgtgttcgtgtctccatctcacgccgctgagtctaagtacggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggccctgcacaatcactacacccagaagtccctgtctctgtctcttggcggaggcggaggatctgctcctacctccagctccaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacctccagtgcctggaagaggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagtccatcatctccacactgacc

SEQ ID NO: 19 - protein, 367 aa, synthetic construct
  REGION 1..367: note = fusion protein (GI101C2)
AESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 20 - DNA, 1434 bp, synthetic construct
  misc_feature 1..1434: note = nucleotiedes coding fusion protein (mGI101C1)
  source 1..1434: mol_type = other DNA
atggatgctatgctgagaggcctgtgttgcgtgctgctgctgtgtggcgctgtgttcgtgtctccttctcacgctgtggacgagcagctctccaagtccgtgaaggataaggtcctgctgccttgccggtacaactctcctcacgaggacgagtctgaggaccggatctactggcagaaacacgacaaggtggtgctgtccgtgatcgccggaaagctgaaagtgtggcctgagtacaagaacaggaccctgtacgacaacaccacctacagcctgatcatcctgggcctcgtgctgagcgatagaggcacctattcttgcgtggtgcagaagaaagagcggggcacctacgaagtgaagcacctggctctggtcaagctgtccatcaaggccgacttcagcacccctaacatcaccgagtctggcaacccttccgccgacaccaagagaatcacctgtttcgcctctggcggcttccctaagcctcggttctcttggctggaaaacggcagagagctgcccggcatcaataccaccatttctcaggacccagagtccgagctgtacaccatctccagccagctcgactttaacaccaccagaaaccacaccatcaagtgcctgattaagtacggcgacgcccacgtgtccgaggactttacttgggagaaacctcctgaggaccctcctgactctggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctatagagtggtgtccgtgctgaccgtgctgcaccaggattggctgaacggcaaagagtacaagtgcaaggtgtccaacaagggcctgccttccagcatcgaaaagaccatcagcaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggctctgcacaaccactacacccagaagtccctgtctctgtccctgggc

SEQ ID NO: 21 - protein, 478 aa, synthetic construct
  REGION 1..478: note = fusion protein (mGI101C1)
MDAMLRGLCCVLLLCGAVFVSPSHAVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 22 - protein, 133 aa, synthetic construct
  REGION 1..133: note = variants of IL-2 (3M, M45)
APTSSSTKKTQLQLEHLLLDLQMILNGINNYKNPKLTAMLTAKFAMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 133 aa, synthetic construct
  REGION 1..133: note = variants of IL-2 (3M, M61)
APTSSSTKKTQLQLEHLLLDLQMILNGINNYKNPKLTAMLTAKFYMPKKATELKHLQCLERELKPLEEVLNLAQSKNFHLRPRDLISNINVIVLELKGSETTFMCEYADETATIVEFLNRWITFCQSIISTLT

SEQ ID NO: 24 - protein, 133 aa, synthetic construct
  REGION 1..133: note = variants of IL-2 (3M, M72)
APTSSSTKKTQLQLEHLLLDLQMILNGINNYKNPKLTAMLTAKFYMPKKATELKHLQCLEEELKPLEEVLNGAQSKNFHLRPRDLISNINVIVLELKGSETTFMCEYADETATIVEFLNRWITFCQSIISTLT

SEQ ID NO: 25 - DNA, 1851 bp, synthetic construct
  misc_feature 1..1851: note = nucleotiedes coding fusion protein (GI102-M45)
  source 1..1851: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttcgccatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 26 - protein, 592 aa, synthetic construct
  REGION 1..592: note = fusion protein (GI102-M45)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFAMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - DNA, 1851 bp, synthetic construct
  misc_feature 1..1851: note = nucleotiedes coding fusion protein (GI102-M61)
  source 1..1851: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttctacatgcccaagaaggccaccgagctgaagcacctccagtgcctggaaagggaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 28 - protein, 592 aa, synthetic construct
  REGION 1..592: note = fusion protein (GI102-M61)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLERELKPLEEVLNLAQSKNFHLRPRDLISNINVIVLELKGSETTFMCEYADETATIVEFLNRWITFCQSIISTLT

SEQ ID NO: 29 - DNA, 1857 bp, synthetic construct
  misc_feature 1..1857: note = nucleotiedes coding fusion protein (GI102-M72)
  source 1..1857: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttctacatgcccaagaaggccaccgagctgaagcacctccagtgcctggaagaagaactgaagcccctggaagaggtgctgaatggggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacctgatga

SEQ ID NO: 30 - protein, 592 aa, synthetic construct
  REGION 1..592: note = fusion protein (GI102-M72)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNGAQSKNFHLRPRDLISNINVIVLELKGSETTFMCEYADETATIVEFLNRWITFCQSIISTLT

SEQ ID NO: 31 - DNA, 1851 bp, synthetic construct
  misc_feature 1..1851: note = nucleotiedes coding fusion protein (GI101w)
  source 1..1851: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgacccgcatgctgacctttaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 32 - protein, 592 aa, synthetic construct
  REGION 1..592: note = fusion protein (GI101w)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 33 - DNA, 1848 bp, synthetic construct
  misc_feature 1..1848: note = nucleotiedes coding fusion protein (mGI102-M61)
  source 1..1848: mol_type = other DNA
atggatgctatgctgagaggcctgtgttgcgtgctgctgctgtgtggcgctgtgttcgtgtctccttctcacgctgtggacgagcagctctccaagtccgtgaaggataaggtcctgctgccttgccggtacaactctcctcacgaggacgagtctgaggaccggatctactggcagaaacacgacaaggtggtgctgtccgtgatcgccggaaagctgaaagtgtggcctgagtacaagaacaggaccctgtacgacaacaccacctacagcctgatcatcctgggcctcgtgctgagcgatagaggcacctattcttgcgtggtgcagaagaaagagcggggcacctacgaagtgaagcacctggctctggtcaagctgtccatcaaggccgacttcagcacccctaacatcaccgagtctggcaacccttccgccgacaccaagagaatcacctgtttcgcctctggcggcttccctaagcctcggttctcttggctggaaaacggcagagagctgcccggcatcaataccaccatttctcaggacccagagtccgagctgtacaccatctccagccagctcgactttaacaccaccagaaaccacaccatcaagtgcctgattaagtacggcgacgcccacgtgtccgaggactttacttgggagaaacctcctgaggaccctcctgactctggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctatagagtggtgtccgtgctgaccgtgctgcaccaggattggctgaacggcaaagagtacaagtgcaaggtgtccaacaagggcctgccttccagcatcgaaaagaccatcagcaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggctctgcacaaccactacacccagaagtccctgtctctgtctcttggaggtggtggcggttctgcccctacctccagctctaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacttgcagtgcctggaaagggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagagcatcatctccacactgacc

SEQ ID NO: 34 - protein, 616 aa, synthetic construct
  REGION 1..616: note = fusion protein (mGI102-M61)
MDAMLRGLCCVLLLCGAVFVSPSHAVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLERELKPLEEVLNLAQSKNFHLRPRDLISNINVIVLELKGSETTFMCEYADETATIVEFLNRWITFCQSIISTLT

SEQ ID NO: 35 - protein, 153 aa, synthetic construct
  REGION 1..153: note = wild type hIL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 36 - protein, 158 aa, synthetic construct
  REGION 1..158: note = IL-2 with signal sequence
MDAMLRGLCCVLLLCGAVFVSPSHAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 37 - DNA, 474 bp, synthetic construct
  misc_feature 1..474: note = nucleotide sequence coding IL-2 with signal sequence
  source 1..474: mol_type = other DNA
atggatgctatgctgagaggcctgtgttgcgtgctgctgctgtgtggcgctgtgttcgtgtctccttctcacgctgcccctaccagctcctctaccaagaaaacccagctccagttggagcatctgctgctggacctccagatgattctgaacgggatcaacaactataagaaccccaagctgacccgcatgctgacctttaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 38 - protein, 591 aa, synthetic construct
  REGION 1..591: note = mGI-101
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

(19) United States
(12) Patent Application Publication
(10) Pub. No.: US 2026/0240769 A1
(43) Pub. Date: Aug. 20, 2026

(54) FUSION PROTEIN PREPARATION COMPRISING IL-2 AND CD80 PROTEINS

(71) Applicant: GI INNOVATION, INC., Seoul (KR)

(72) Inventors: Myung Ho JANG, Seoul (KR); Young Min OH, Yongin-si (KR); Heonchang LIM, Incheon (KR)

(73) Assignee: GI INNOVATION, INC., Seoul (KR)

(21) Appl. No.: 19/665,445

(22) Filed: May 1, 2026

Related U.S. Application Data
(63) Continuation of application No. 17/912,398, filed on Mar. 17, 2021, now Pat. No. 12,642,764.

(30) Foreign Application Priority Data
Mar. 18, 2020 (KR) 10-2020-0033231

(51) Int. Cl. A61K 9/08; A61K 38/17; A61K 38/20; A61K 47/10; A61K 47/18; A61K 47/22; A61K 47/26

(52) U.S. Cl. CPC A61K 9/08; A61K 38/1774; A61K 38/2013; A61K 47/10; A61K 47/183; A61K 47/22; A61K 47/26

(57) ABSTRACT

The present invention relates to a pharmaceutical formulation with enhanced stability of a fusion protein dimer comprising a modified IL-2 protein and a CD80 protein. The fusion protein dimer comprising an IL-2 protein and a CD80 protein can not only activate immune cells owing to IL-2, but also effectively regulate Treg cells owing to CD80. When the formulation according to the present invention is applied to a fusion protein dimer comprising an IL-2 protein and a CD80 protein, the stability of the fusion protein dimer is significantly increased, and it can be used as a liquid formulation. Accordingly, the commercial applicability of the fusion protein dimer can be increased.

### CROSS REFERENCE TO RELATED APPLICATIONS

This application is a Continuation of U.S. application Ser. No. 17/912,398 filed Sep. 16, 2022, which is a National Stage of International Application No. PCT/KR2021/003312 filed Mar. 17, 2021, claiming priority based on Korean Patent Application No. 10-2020-0033231 filed Mar. 18, 2020.

### INCORPORATION BY REFERENCE OF SEQUENCE LISTING

The content of the electronically submitted sequence listing, file name: Q320091SEQLIST.xml; size: 74,977 bytes; and date of creation: May 1, 2026, filed herewith, is incorporated herein by reference in its entirety.

### TECHNICAL FIELD

The present invention relates to a liquid formulation with enhanced stability of a fusion protein comprising an IL-2 protein and a CD80 protein.

### BACKGROUND ART

IL-2, also called T-cell growth factor, is a globular glycoprotein that plays a central role in lymphocyte production, survival, and homeostasis. IL-2 has a protein size of about 15.5 kDa to about 16 kDa and consists of 133 amino acids. IL-2 mediates various immune actions by binding to an IL-2 receptor composed of three distinct subunits. In addition, IL-2 is synthesized mainly by activated T cells, in particular by CD4+ helper T cells. IL-2 stimulates proliferation and differentiation of T cells, and induces production of cytotoxic T lymphocytes and differentiation of peripheral blood lymphocytes into cytotoxic cells and lymphokine-activated killer cells.

Furthermore, IL-2 is involved in proliferation and differentiation of B cells, and promotes immunoglobulin synthesis by B cells. In addition, IL-2 stimulates production, proliferation, and activation of natural killer cells. Therefore, IL-2 is used as an anticancer agent, because it can increase lymphocyte populations and increase the function of the immune cells in vivo. Currently, therapy with IL-2 has been approved for patients with metastatic renal cell carcinoma and malignant melanoma.

However, IL-2 has a dual function in that it is important not only for mediating an increase in number of immune cells and activity thereof, but also for maintaining immune tolerance. In addition, it has been reported that IL-2 may not be optimal for inhibiting tumor growth. The reason is that in the presence of IL-2, activation-induced cell death (AICD) may occur in the resulting cytotoxic T lymphocytes and immune responses may be inhibited by IL-2-dependent regulatory T cells (Treg cells) (Imai et al., Cancer Sci 98, 416-423, 2007).

In addition, severe cardiovascular, pulmonary, renal, hepatic, gastrointestinal, neuronal, dermatological, hematological, and systemic side effects occur in patients who have received IL-2. Therefore, various IL-2 mutants have been studied to improve therapeutic efficacy of IL-2 and minimize side effects thereof (U.S. Pat. No. 5,229,109 B). However, there are still many problems to be solved in order to utilize IL-2 for pharmacological purposes.

Meanwhile, CD80, also known as B7-1, is a member of the B7 family of membrane-bound proteins that are involved in immune regulation by binding to its ligand by way of delivering costimulatory responses and coinhibitory responses. CD80 is a transmembrane protein expressed on the surface of T cells, B cells, dendritic cells, and monocytes. CD80 is known to bind CD28, CTLA4 (CD152), and PD-L1. CD80, CD86, CTLA4, and CD28 are involved in a costimulatory-coinhibitory system. For example, CD80 is known to regulate activity of T cells and to be involved in proliferation, differentiation, and survival thereof.

For example, when CD80 and CD86 interact with CD28, costimulatory signals are generated to activate T cells. Eventually, CD80 binds to CTLA4 expressed on the surface of activated T cells and stimulates CTLA4 to be upregulated. As a result, CD80 inhibits T cell responses prior to immune response operation caused by CD80/CD28 interaction. This feedback loop allows for fine regulation of immune responses.

In addition, CD80 is known to bind PD-L 1, another B7 family member, with affinity similar to that with which CD28 binds PD-L1. PD-L1 is known as one of two ligands for programmed death-1 (PD-1) protein, and PD-L1 is known to be involved in T cell regulation. Binding of CD80 to PD-L1 is another mechanism that can block PD-1/PD-L1 interaction, which may prevent inhibition of T cell responses in tumors. However, an increase in CD80 levels causes CD80 to bind to CD28, thereby inducing T cell responses. At the same time, CD80 may bind to CTLA4, thereby inhibiting T cell responses.

It was confirmed that a fusion protein comprising a CD80 fragment, an immunoglobulin Fe, and an IL-2 variant can activate immune cells, and at the same time, can control the immune cell regulatory activity of regulatory T cells, and thus can efficiently treat cancer as well as an infectious disease (KR 10-2201086 B1). In order to efficiently apply such protein to the treatment of cancer disease and an infectious disease, it is necessary to develop a stable, high-concentration protein formulation that provides dosing and administration advantages.

### DETAILED DESCRIPTION OF INVENTION

### Technical Problem

The present inventors developed a formulation with increased stability of a novel fusion protein dimer comprising, in one molecule, an IL-2 protein and a CD80 protein, thereby completing the present invention.

### Solution to Problem

In order to achieve the above object, in an aspect of the present invention, there is provided a pharmaceutical formulation comprising a fusion protein dimer comprising an IL-2 protein and a CD80 protein.

### Effects of Invention

A fusion protein dimer comprising an IL-2 protein and a CD80 protein can not only activate immune cells owing to IL-2, but also effectively regulate Treg cells owing to CD80. In order to use such a fusion protein dimer clinically, the stability of the protein preparation must be secured. When the pharmaceutical formulation according to the present invention is applied to a fusion protein dimer comprising an IL-2 protein and a CD80 protein, the stability of the fusion protein dimer is significantly increased, and it can be used as a liquid formulation.

Accordingly, the commercial applicability of the fusion protein dimer can be increased.

### BRIEF DESCRIPTION OF DRAWINGS

FIG. 1 illustrates a result detecting the obtained fusion protein (GI-101) with SDS-PAGE.

FIG. 2 illustrates amounts of the fusion protein (GI-101) depending on absorbance.

FIG. 3 illustrates a result analyzing the obtained fusion protein (GI-101) by size exclusion chromatography (SEC).

### BEST MODE FOR CARRYING OUT THE INVENTION

### Pharmaceutical Formulation Comprising Fusion Protein Comprising IL-2 Protein And CD80 Protein

In an aspect of the present invention, there is provided a pharmaceutical formulation comprising: (i) a fusion protein dimer comprising an IL-2 protein and a CD80 protein at a concentration of 3.0 mg/mL to 5.0 mg/mL; (ii) a buffer at a concentration of 10 mM to 30 mM; and (iii) a surfactant at a concentration of 0.155 w/w % to 0.185 w/w %; wherein the pH of the formulation is from 6.5 to 7.5.

Here, the pharmaceutical formulation may be a liquid formulation.

### Fusion Protein Comprising IL-2 Protein and CD80 Protein

As used herein, the term “IL-2” or “interleukin-2”, unless otherwise stated, refers to any wild-type IL-2 obtained from any vertebrate source, including mammals, for example, primates (such as humans) and rodents (such as mice and rats). IL-2 may be obtained from animal cells, and also includes one obtained from recombinant cells capable of producing IL-2. In addition, IL-2 may be wild-type IL-2 or a variant thereof.

In the present specification, IL-2 or a variant thereof may be collectively expressed by the term “IL-2 protein” or “IL-2 polypeptide.” IL-2, an IL-2 protein, an IL-2 polypeptide, and an IL-2 variant specifically bind to, for example, an IL-2 receptor. This specific binding may be identified by methods known to those skilled in the art.

An embodiment of IL-2 may have the amino acid sequence of SEQ ID NO: 35 or SEQ ID NO: 36. Here, IL-2 may also be in a mature form. Specifically, the mature IL-2 may not contain a signal sequence, and may have the amino acid sequence of SEQ ID NO: 10. Here, IL-2 may be used under a concept encompassing a fragment of wild-type IL-2 in which a portion of N-terminus or C-terminus of the wild-type IL-2 is truncated.

In addition, the fragment of IL-2 may be in a form in which 1, 2, 3, 4, 5, 6, 7, 8, 9, 10, 11, 12, 13, 14, 15, 16, 17, 18, 19, 20, 21, 22, 23, 24, or 25 contiguous amino acids are truncated from N-terminus of a protein having the amino acid sequence of SEQ ID NO: 35 or SEQ ID NO: 36. In addition, the fragment of IL-2 may be in a form in which 1, 2, 3, 4, 5, 6, 7, 8, 9, 10, 11, 12, 13, 14, 15, 16, 17, 18, 19, 20, 21, 22, 23, 24, or 25 contiguous amino acids are truncated from C-terminus of a protein having the amino acid sequence of SEQ ID NO: 35 or SEQ ID NO: 36.

As used herein, the term “IL-2 variant” refers to a form in which a portion of amino acids in the full-length IL-2 or the above-described fragment of IL-2 is substituted. That is, an IL-2 variant may have an amino acid sequence different from wild-type IL-2 or a fragment thereof. However, an IL-2 variant may have activity equivalent or similar to the wild-type IL-2. Here, “IL-2 activity” may, for example, refer to specific binding to an IL-2 receptor, which specific binding can be measured by methods known to those skilled in the art.

Specifically, an IL-2 variant may be obtained by substitution of a portion of amino acids in the wild-type IL-2. An embodiment of the IL-2 variant obtained by amino acid substitution may be obtained by substitution of at least one of the 38^{th}, 42^{nd}, 45^{th}, 61^{st}, and 72^{nd} amino acids in the amino acid sequence of SEQ ID NO: 10.

Specifically, the IL-2 variant may be obtained by substitution of at least one of the 38^{th}, 42^{nd}, 45^{th}, 61^{st}, or 72^{nd} amino acid in the amino acid sequence of SEQ ID NO: 10 with another amino acid. In addition, when IL-2 is in a form in which a portion of N-terminus in the amino acid sequence of SEQ ID NO: 35 is truncated, the amino acid at a position complementarily corresponding to that in the amino acid sequence of SEQ ID NO: 10 may be substituted with another amino acid. For example, when IL-2 has the amino acid sequence of SEQ ID NO: 35, its IL-2 variant may be obtained by substitution of at least one of 58^{th}, 62^{nd}, 65^{th}, 81^{st}, or 92^{nd} amino acid in the amino acid sequence of SEQ ID NO: 35 with another amino acid. These amino acid residues correspond to the 38^{th}, 42^{nd}, 45^{th}, 61^{st}, and 72^{nd} amino acid residues in the amino acid sequence of SEQ ID NO: 10, respectively. According to an embodiment, one, two, three, four, five, six, seven, eight, nine, or ten amino acids may be substituted as long as such IL-2 variant maintains IL-2 activity. According to another embodiment, one to five amino acids may be substituted.

In an embodiment, an IL-2 variant may be in a form in which two amino acids are substituted. Specifically, the IL-2 variant may be obtained by substitution of the 38^{th} and 42^{nd} amino acids in the amino acid sequence of SEQ ID NO: 10. In addition, in an embodiment, the IL-2 variant may be obtained by substitution of the 38^{th} and 45^{th} amino acids in the amino acid sequence of SEQ ID NO: 10. In addition, in an embodiment, the IL-2 variant may be obtained by substitution of the 38^{th} and 61^{st} amino acids in the amino acid sequence of SEQ ID NO: 10. In addition, in an embodiment, the IL-2 variant may be obtained by substitution of the 38^{th} and 72^{nd} amino acids in the amino acid sequence of SEQ ID NO: 10. In addition, in an embodiment, the IL-2 variant may be obtained by substitution of the 42^{nd} and 45^{th} amino acids in the amino acid sequence of SEQ ID NO: 10. In addition, in an embodiment, the IL-2 variant may be obtained by substitution of the 42^{nd} and 61^{st} amino acids in the amino acid sequence of SEQ ID NO: 10. In addition, in an embodiment, the IL-2 variant may be obtained by substitution of the 42^{nd} and 72^{nd} amino acids in the amino acid sequence of SEQ ID NO: 10. In addition, in an embodiment, the IL-2 variant may be obtained by substitution of the 45^{th} and 61^{st} amino acids in the amino acid sequence of SEQ ID NO: 10. In addition, in an embodiment, the IL-2 variant may be obtained by substitution of the 45^{th} and 72^{nd} amino acids in the amino acid sequence of SEQ ID NO: 10. In addition, in an embodiment, the IL-2 variant may be obtained by substitution of the 61^{st} and 72^{nd} amino acids in the amino acid sequence of SEQ ID NO: 10.

Furthermore, an IL-2 variant may be in a form in which three amino acids are substituted. Specifically, the IL-2 variant may be obtained by substitution of the 38^{th}, 42^{nd}, and 45^{th} amino acids in the amino acid sequence of SEQ ID NO: 10. In addition, in an embodiment, the IL-2 variant may be obtained by substitution of the 38^{th}, 42^{nd}, and 61^{st} amino acids in the amino acid sequence of SEQ ID NO: 10. In addition, in an embodiment, the IL-2 variant may be obtained by substitution of the 38^{th}, 42^{nd}, and 72^{nd} amino acids in the amino acid sequence of SEQ ID NO: 10. In addition, in an embodiment, the IL-2 variant may be obtained by substitution of the 38^{th}, 45^{th}, and 61^{st} amino acids in the amino acid sequence of SEQ ID NO: 10. In addition, in an embodiment, the IL-2 variant may be obtained by substitution of the 38^{th}, 45^{th}, and 72^{nd} amino acids in the amino acid sequence of SEQ ID NO: 10. In addition, in an embodiment, the IL-2 variant may be obtained by substitution of the 38^{th}, 61^{st}, and 72^{nd} amino acids in the amino acid sequence of SEQ ID NO: 10. In addition, in an embodiment, the IL-2 variant may be obtained by substitution of the 42^{nd}, 45^{th}, and 61^{st} amino acids in the amino acid sequence of SEQ ID NO: 10. In addition, in an embodiment, the IL-2 variant may be obtained by substitution of the 42^{nd}, 45^{th}, and 72^{nd} amino acids in the amino acid sequence of SEQ ID NO: 10. In addition, in an embodiment, the IL-2 variant may be obtained by substitution of the 45^{th}, 61^{st}, and 72^{nd} amino acids in the amino acid sequence of SEQ ID NO: 10.

In addition, an IL-2 variant may be in a form in which four amino acids are substituted. Specifically, the IL-2 variant may be obtained by substitution of the 38^{th}, 42^{nd}, 45^{th}, and 61^{st} amino acids in the amino acid sequence of SEQ ID NO: 10. In addition, in an embodiment, the IL-2 variant may be obtained by substitution of the 38^{th}, 42^{nd}, 45^{th}, and 72^{nd} amino acids in the amino acid sequence of SEQ ID NO: 10. In addition, in an embodiment, the IL-2 variant may be obtained by substitution of the 38^{th}, 45^{th}, 61^{st}, and 72^{nd} amino acids in the amino acid sequence of SEQ ID NO: 10. In addition, in an embodiment, the IL-2 variant may be obtained by substitution of the 38^{th}, 42^{nd}, 61^{st}, and 72^{nd} amino acids in the amino acid sequence of SEQ ID NO: 10. In addition, in an embodiment, the IL-2 variant may be obtained by substitution of 42^{nd}, 45^{th}, 61^{st}, and 72^{nd} amino acids in the amino acid sequence of SEQ ID NO: 10.

Furthermore, an IL-2 variant may be in a form in which five amino acids are substituted. Specifically, the IL-2 variant may be obtained by substitution of each of the 38^{th}, 42^{nd}, 45^{th}, 61^{st}, and 72^{nd} amino acids in the amino acid sequence of SEQ ID NO: 10 with another amino acid.

Here, the “another amino acid” introduced by the substitution may be any one selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. However, regarding amino acid substitution for the IL-2 variant, in the amino acid sequence of SEQ ID NO: 10, the 38^{th} amino acid cannot be substituted with arginine, the 42^{nd} amino acid cannot be substituted with phenylalanine, the 45^{th} amino acid cannot be substituted with tyrosine, the 61^{st} amino acid cannot be substituted with glutamic acid, and the 72^{nd} amino acid cannot be substituted with leucine.

Regarding amino acid substitution for an IL-2 variant, in the amino acid sequence of SEQ ID NO: 10, the 38^{th} amino acid, arginine, may be substituted with an amino acid other than arginine. Preferably, regarding amino acid substitution for an IL-2 variant, in the amino acid sequence of SEQ ID NO: 10, the 38^{th} amino acid, arginine, may be substituted with alanine (R38A).

Regarding amino acid substitution for an IL-2 variant, in the amino acid sequence of SEQ ID NO: 10, the 42^{nd} amino acid, phenylalanine, may be substituted with an amino acid other than phenylalanine. Preferably, regarding amino acid substitution for an IL-2 variant, in the amino acid sequence of SEQ ID NO: 10, the 42^{nd} amino acid, phenylalanine, may be substituted with alanine (F42A).

Regarding amino acid substitution for an IL-2 variant, in the amino acid sequence of SEQ ID NO: 10, the 45^{th} amino acid, tyrosine, may be substituted with an amino acid other than tyrosine. Preferably, regarding amino acid substitution for an IL-2 variant, in the amino acid sequence of SEQ ID NO: 10, the 45^{th} amino acid, tyrosine, may be substituted with alanine (Y45A).

Regarding amino acid substitution for an IL-2 variant, in the amino acid sequence of SEQ ID NO: 10, the 61^{st} amino acid, glutamic acid, may be substituted with an amino acid other than glutamic acid. Preferably, regarding amino acid substitution for an IL-2 variant, in the amino acid sequence of SEQ ID NO: 10, the 61^{st} amino acid, glutamic acid, may be substituted with arginine (E61R).

Regarding amino acid substitution for an IL-2 variant, in the amino acid sequence of SEQ ID NO: 10, the 72^{nd} amino acid, leucine, may be substituted with an amino acid other than leucine. Preferably, regarding amino acid substitution for an IL-2 variant, in the amino acid sequence of SEQ ID NO: 10, the 72^{nd} amino acid, leucine, may be substituted with glycine (L72G).

Specifically, an IL-2 variant may be obtained by at least one substitution selected from the group consisting of R38A, F42A, Y45A, E61R, and L72G, in the amino acid sequence of SEQ ID NO: 10.

Specifically, an IL-2 variant may be obtained by amino acid substitutions at two, three, four, or five positions among the positions selected from the group consisting of R38A, F42A, Y45A, E61R, and L72G.

In addition, an IL-2 variant may be in a form in which two amino acids are substituted. Specifically, an IL-2 variant may be obtained by the substitutions, R38A and F42A. In addition, in an embodiment, an IL-2 variant may be obtained by the substitutions, R38A and Y45A. In addition, in an embodiment, an IL-2 variant may be obtained by the substitutions, R38A and E61R. In addition, in an embodiment, an IL-2 variant may be obtained by the substitutions, R38A and L72G. In addition, in an embodiment, an IL-2 variant may be obtained by the substitutions, F42A and Y45A. In addition, in an embodiment, an IL-2 variant may be obtained by the substitutions, F42A and E61R. In addition, in an embodiment, an IL-2 variant may be obtained by the substitutions, F42A and L72G. In addition, in an embodiment, an IL-2 variant may be obtained by the substitutions, E61R and L72G.

Furthermore, an IL-2 variant may be in a form in which three amino acids are substituted. Specifically, an IL-2 variant may be obtained by the substitutions, R38A, F42A, and Y45A. In addition, in an embodiment, an IL-2 variant may be obtained by the substitutions, R38A, F42A, and E61R. In addition, in an embodiment, an IL-2 variant may be obtained by the substitutions, R38A, F42A, and L72G. In addition, in an embodiment, an IL-2 variant may be obtained by the substitutions, R38A, Y45A, and E61R. In addition, in an embodiment, an IL-2 variant may be obtained by the substitutions, R38A, Y45A, and L72G. In addition, in an embodiment, an IL-2 variant may be obtained by the substitutions, F42A, Y45A, and E61R. In addition, in an embodiment, an IL-2 variant may be obtained by the substitutions, F42A, Y45A, and L72G. In addition, in an embodiment, an IL-2 variant may be obtained by the substitutions, F42A, E61R, and L72G. In addition, in an embodiment, an IL-2 variant may be obtained by the substitutions, Y45A, E61R, and L72G.

In addition, an IL-2 variant may be in a form in which four amino acids are substituted. Specifically, an IL-2 variant may be obtained by the substitutions, R38A, F42A, Y45A, and E61R. In addition, in an embodiment, an IL-2 variant may be obtained by the substitutions, R38A, F42A, Y45A, and L72G. In addition, in an embodiment, an IL-2 variant may be obtained by the substitutions, R38A, F42A, E61R, and L72G. In addition, in an embodiment, an IL-2 variant may be obtained by the substitutions, R38A, Y45A, E61R, and L72G. In addition, in an embodiment, an IL-2 variant may be obtained by the substitutions, F42A, Y45A, E61R, and L72G.

Furthermore, an IL-2 variant may be obtained by the substitutions, R38A, F42A, Y45A, E61R, and L72G.

Preferably, an embodiment of the IL-2 variant may contain which are any one selected from the following substitution combinations (a) to (d) in the amino acid sequence of SEQ ID NO: 10:
(a) R38A/F42A
(b) R38A/F42A/Y45A
(c) R38A/F42A/E61R
(d) R38A/F42A/L72G

Here, when IL-2 has the amino acid sequence of SEQ ID NO: 35, an amino acid substitution may be present at a position complementarily corresponding to that in the amino acid sequence of SEQ ID NO: 10. In addition, even when IL-2 is a fragment of the amino acid sequence of SEQ ID NO: 35, an amino acid substitution may be present at a position complementarily corresponding to that in the amino acid sequence of SEQ ID NO: 10.

Specifically, an IL-2 variant may have the amino acid sequence of SEQ ID NO: 6, 22, 23, or 24.

In addition, an IL-2 variant may be characterized by having low in vivo toxicity. Here, the low in vivo toxicity may be a side effect caused by binding of IL-2 to the IL-2 receptor alpha chain (IL-2Rα). Various IL-2 variants have been developed to ameliorate the side effect caused by binding of IL-2 to IL-2Rα, and such IL-2 variants may be those disclosed in U.S. Pat. No. 5,229,109 and Korean Patent No. 1667096. In particular, IL-2 variants described in the present application have low binding ability for the IL-2 receptor alpha chain (IL-2Rα) and thus have lower in vivo toxicity than the wild-type IL-2.

As used herein, the term “CD80”, also called “B7-1”, is a membrane protein present in dendritic cells, activated B cells, and monocytes. CD80 provides co-stimulatory signals essential for activation and survival of T cells. CD80 is known as a ligand for the two different proteins, CD28 and CTLA-4, present on the surface of T cells. CD80 is composed of 288 amino acids, and may specifically have the amino acid sequence of SEQ ID NO: 11. In addition, as used herein, the term “CD80 protein” refers to the full-length CD80 or a CD80 fragment.

As used herein, the term “CD80 fragment” refers to a cleaved form of CD80. In addition, the CD80 fragment may be an extracellular domain of CD80. An embodiment of the CD80 fragment may be obtained by elimination of the 1^{st} to 34^{th} amino acids from N-terminus which are a signal sequence of CD80. Specifically, an embodiment of the CD80 fragment may be a protein composed of the 35^{th} to 288^{th} amino acids in SEQ ID NO: 11. In addition, an embodiment of the CD80 fragment may be a protein composed of the 35^{th} to 242^{nd} amino acids in SEQ ID NO: 11. In addition, an embodiment of the CD80 fragment may be a protein composed of the 35^{th} to 232^{nd} amino acids in SEQ ID NO: 11. In addition, an embodiment of the CD80 fragment may be a protein composed of the 35^{th} to 139^{th} amino acids in SEQ ID NO: 11. In addition, an embodiment of the CD80 fragment may be a protein composed of the 142^{nd} to 242^{nd} amino acids in SEQ ID NO: 11. In an embodiment, a CD80 fragment may have the amino acid sequence of SEQ ID NO: 2.

In addition, the IL-2 protein and the CD80 protein may be attached to each other via a linker or a carrier. Specifically, the IL-2 or a variant thereof and the CD80 (B7-1) or a fragment thereof may be attached to each other via a linker or a carrier. In the present description, the linker and the carrier may be used interchangeably.

The linker links two proteins. An embodiment of the linker may include 1 to 50 amino acids, albumin or a fragment thereof, an Fc domain of an immunoglobulin, or the like. Here, the Fc domain of immunoglobulin refers to a protein that contains heavy chain constant region 2 (CH2) and heavy chain constant region 3 (CH3) of an immunoglobulin, and does not contain heavy and light chain variable regions and light chain constant region 1 (CH1) of an immunoglobulin. The immunoglobulin may be IgG, IgA, IgE, IgD, or IgM, and may preferably be IgG4. Here, Fc domain of wild-type immunoglobulin G4 may have the amino acid sequence of SEQ ID NO: 4.

In addition, the Fc domain of an immunoglobulin may be an Fc domain variant as well as wild-type Fc domain. In addition, as used herein, the term “Fc domain variant” may refer to a form which is different from the wild-type Fc domain in terms of glycosylation pattern, has a high glycosylation as compared with the wild-type Fc domain, or has a low glycosylation as compared with the wild-type Fc domain, or a deglycosylated form. In addition, an aglycosylated Fc domain is included therein. The Fc domain or a variant thereof may be adapted to have an adjusted number of sialic acids, fucosylations, or glycosylations, through culture conditions or genetic manipulation of a host.

In addition, glycosylation of the Fc domain of an immunoglobulin may be modified by conventional methods such as chemical methods, enzymatic methods, and genetic engineering methods using microorganisms. In addition, the Fc domain variant may be in a mixed form of respective Fe regions of immunoglobulins, IgG, IgA, IgE, IgD, and IgM. In addition, the Fc domain variant may be in a form in which some amino acids of the Fc domain are substituted with other amino acids. An embodiment of the Fc domain variant may have the amino acid sequence of SEQ ID NO: 12.

The fusion protein may have a structure in which, using an Fc domain as a linker (or carrier), a CD80 protein and an IL-2 protein, or an IL-2 protein and a CD80 protein are linked to N-terminus and C-terminus of the linker or carrier, respectively. Linkage between N-terminus or C-terminus of the Fc domain and CD-80 or IL-2 may optionally be achieved by a linker peptide.

Specifically, a fusion protein may consist of the following structural formula (I) or (II):

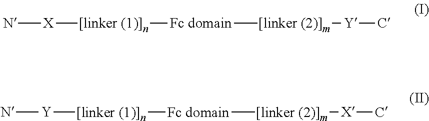

Here, in the structural formulas (I) and (II),
N′ is the N-terminus of the fusion protein,
C′ is the C-terminus of the fusion protein,
X is a CD80 protein,
Y is an IL-2 protein,
the linkers (1) and (2) are peptide linkers, and
n and m are each independently 0 or 1.

Preferably, the fusion protein may consist of the structural formula (I). The IL-2 protein is as described above. In addition, the CD80 protein is as described above. According to an embodiment, the IL-2 protein may be an IL-2 variant with one to five amino acid substitutions as compared with the wild-type IL-2. The CD80 protein may be a fragment obtained by truncation of up to about 34 contiguous amino acid residues from the N-terminus or C-terminus of the wild-type CD80. Alternatively, the CD protein may be an extracellular immunoglobulin-like domain having the activity of binding to the T cell surface receptors CTLA-4 and CD28.

Specifically, the fusion protein may have the amino acid sequence of SEQ ID NO: 9, 26, 28, or 30. According to another embodiment, the fusion protein includes a polypeptide having a sequence identity of 85%, 86%, 87%, 88%, 89%, 90%, 91%, 92%, 93%, 94%, 95%, 96%, 97%, 98%, 99%, or 100% to the amino acid sequence of SEQ ID NO: 9, 26, 28, or 30. Here, the identity is, for example, percent homology, and may be determined through homology comparison software such as BlastN software of the National Center of Biotechnology Information (NCBI).

The peptide linker (1) may be included between the CD80 protein and the Fc domain. The peptide linker (1) may consist of 5 to 80 contiguous amino acids, 20 to 60 contiguous amino acids, 25 to 50 contiguous amino acids, or 30 to 40 contiguous amino acids. In an embodiment, the peptide linker (1) may consist of 30 amino acids. In addition, the peptide linker (1) may contain at least one cysteine. Specifically, the peptide linker (1) may contain one, two, or three cysteines. In addition, the peptide linker (1) may be derived from the hinge of an immunoglobulin. In an embodiment, the peptide linker (1) may be a peptide linker consisting of the amino acid sequence of SEQ ID NO: 3.

The peptide linker (2) may consist of 1 to 50 contiguous amino acids, 3 to 30 contiguous amino acids, or 5 to 15 contiguous amino acids. In an embodiment, the peptide linker (2) may be (G4S)ₙ (where n is an integer of 1 to 10). Here, in (G4S)ₙ, n may be 1, 2, 3, 4, 5, 6, 7, 8, 9, or 10. In an embodiment, the peptide linker (2) may be a peptide linker consisting of the amino acid sequence of SEQ ID NO: 5.

In another aspect of the present invention, there is provided a dimer obtained by binding of two fusion proteins, each of which comprises an IL-2 protein and a CD80 protein. The fusion protein comprising IL-2 or a variant thereof and CD80 or a fragment thereof is as described above.

Here, the binding between the fusion proteins constituting the dimer may be achieved by, but is not limited to, a disulfide bond formed by cysteines present in the linker. The fusion proteins constituting the dimer may be the same or different fusion proteins from each other. Preferably, the dimer may be a homodimer. An embodiment of the fusion protein constituting the dimer may be a protein having the amino acid sequence of SEQ ID NO: 9.

### Pharmaceutical Formulation

In an aspect of the present invention, there is provided a pharmaceutical formulation comprising the fusion protein dimer comprising an IL-2 protein and a CD80 protein.

As used herein, the term “pharmaceutical formulation” refers to a preparation that exists in a form that allows the biological activity of an active ingredient to be clearly effective, and does not contain an ingredient that causes side effects in a subject to which the formulation is administered.

The term “subject” may be a mammal such as human, dog, cow, horse, pig, sheep, goat, cat, mouse, rabbit, and rat, and may be preferably human, dog, or cat.

As used herein, the term “pharmaceutical formulation” refers to a pharmaceutical formulation that uses a suitable aqueous solvent, such as water or an aqueous/oily mixture (for example, a water alcohol mixture). The formulation may maintain stability, such as chemical or physical stability, biological activity.

The term “stability” refers to a property that maintains a constant state, and is generally related to minimize degradation, denaturation, aggregation, or unfolding of a biologically active substance, such as a protein, a peptide or a biologically active macromolecule.

Meanwhile, the formulation may be a liquid formulation. The liquid formulation is a aqueous solution or suspension, and may be stably maintained at room temperature, refrigerated (for example, 2° C. to 8° C.) or frozen (for example, −20° C. or −70° C.) during storage.

The pharmaceutical formulation of the present invention may be administered parenterally. Here, parenteral administration may be performed by a method such as subcutaneous administration, intravenous administration, mucosal administration, and intramuscular administration. In an embodiment of the present invention, the formulation may be preferably administered by intravenous injection.

The fusion protein dimer in the pharmaceutical formulation may be at a concentration of 3.0 mg/mL to 5.0 mg/mL. In addition, the fusion protein dimer may be at a concentration of 3.0 mg/mL to 4.8 mg/mL, at a concentration of 3.0 mg/mL to 4.6 mg/mL, at a concentration of 3.0 mg/mL to 4.4 mg/mL, at a concentration of 3.0 mg/mL to 4.2 mg/mL, at a concentration of 3.2 mg/mL to 4.8 mg/mL, at a concentration of 3.2 mg/mL to 4.6 mg/mL, at a concentration of 3.2 mg/mL to 4.4 mg/mL, at a concentration of 3.2 mg/mL to 4.2 mg/mL, at a concentration of 3.4 mg/mL to 4.8 mg/mL, at a concentration of 3.4 mg/mL to 4.6 mg/mL, at a concentration of 3.4 mg/mL to 4.4 mg/mL, at a concentration of 3.4 mg/mL to 4.2 mg/mL, at a concentration of 3.6 mg/mL to 4.8 mg/mL, at a concentration of 3.6 mg/mL to 4.6 mg/mL, at a concentration of 3.6 mg/mL to 4.4 mg/mL, at a concentration of 3.6 mg/mL to 4.2 mg/mL, at a concentration of 3.8 mg/mL to 4.8 mg/mL, at a concentration of 3.8 mg/mL to 4.6 mg/mL, at a concentration of 3.8 mg/mL to 4.4 mg/mL, at a concentration of 3.8 mg/mL to 4.2 mg/mL, or at a concentration of 3.9 mg/mL to 4.1 mg/mL. Specifically, the fusion protein dimer may be at a concentration of 4.0 mg/mL.

In addition, the buffer may be a histidine buffer. Here, the histidine may be at a concentration of 10 mM to 30 mM. In addition, the histidine may be at a concentration of 10 mM to 28 mM, at a concentration of 10 mM to 26 mM, at a concentration of 10 mM to 24 mM, at a concentration of 10 mM to 22 mM, at a concentration of 10 mM to 21 mM, at a concentration of 12 mM to 28 mM, at a concentration of 12 mM to 26 mM, at a concentration of 12 mM to 24 mM, at a concentration of 12 mM to 22 mM, at a concentration of 12 mM to 21 mM, at a concentration of 14 mM to 28 mM, at a concentration of 14 mM to 26 mM, at a concentration of 14 mM to 24 mM, at a concentration of 14 mM to 22 mM, at a concentration of 14 mM to 21 mM, at a concentration of 16 mM to 28 mM, at a concentration of 16 mM to 26 mM, at a concentration of 16 mM to 24 mM, at a concentration of 16 mM to 22 mM, at a concentration of 16 mM to 21 mM, at a concentration of 18 mM to 28 mM, at a concentration of 18 mM to 26 mM, at a concentration of 18 mM to 24 mM, at a concentration of 18 mM to 22 mM, at a concentration of 18 mM to 21 mM, at a concentration of 19 mM to 28 mM, at a concentration of 19 mM to 26 mM, at a concentration of 19 mM to 24 mM, at a concentration of 19 mM to 22 mM, or at a concentration of 19 mM to 21 mM. Specifically, the histidine may be at a concentration of 20 mM.

In addition, the pH of the pharmaceutical formulation may be from 6.5 to 7.5. In addition, the pH of the pharmaceutical formulation may be from 6.5 to 7.3, from 6.5 to 7.2, from 6.5 to 7.1, from 6.7 to 7.3, from 6.7 to 7.2, from 6.7 to 7.1, from 6.8 to 7.3, from 6.8 to 7.2, from 6.8 to 7.1, from 6.9 to 7.3, from 6.9 to 7.2, or from 6.9 to 7.1. Preferably, the pH of the pharmaceutical formulation may be 7.0.

In addition, the surfactant of the pharmaceutical formulation may include any one selected from the group consisting of polysorbate (e.g.: polysorbate 20, polysorbate 28, polysorbate 40, polysorbate 60, polysorbate 65, polysorbate 80, polysorbate 81, and polysorbate 85); poloxamer (e.g.: poloxamer 181, poloxamer 188, and poloxamer 407); polyethylene glycol (PEG); and a combination thereof. Preferably, the pharmaceutical formulation may include two of the surfactants.

In addition, the surfactant may be included at a concentration of 0.065 w/w % to 0.2 w/w % in the formulation. In addition, the surfactant may be included at a concentration of 0.155 w/w % to 0.185 w/w % in the formulation. In an embodiment, the surfactant may be poloxamer 188. Here, the surfactant may be included at a concentration of 0.065 w/w % to 0.075 w/w % in the formulation. In addition, in an embodiment, the surfactant may be polysorbate 80. Here, the surfactant may be included at a concentration of about 0.09 w/w % to about 0.11 w/w % in the formulation. Preferably, poloxamer 188 and polysorbate 80 may be included in the formulation, and they may be included at a concentration of 0.065 w/w % to 0.075 w/w % and at a concentration of 0.09 w/w % to about 0.11 w/w %, respectively. Specifically, poloxamer 188 and polysorbate 80 may be included at a concentration of 0.07 w/w % and 0.1 w/w % in the formulation, respectively.

In addition, the pharmaceutical formulation may further comprise an amino acid. The amino acid may be any one selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan.

Here, the amino acid may be at a concentration of 10 mg/mL to 30 mg/mL. In addition, the amino acid may be at a concentration of 10 mg/mL to 25 mg/mL, at a concentration of 10 mg/mL to 20 mg/mL, at a concentration of 10 mg/mL to 18 mg/mL, at a concentration of 10 mg/mL to 16 mg/mL, at a concentration of 12 mg/mL to 25 mg/mL, at a concentration of 12 mg/mL to 20 mg/mL, at a concentration of 12 mg/mL to 18 mg/mL, at a concentration of 12 mg/mL to 16 mg/mL, at a concentration of 14 mg/mL to 25 mg/mL, at a concentration of 14 mg/mL to 20 mg/mL, at a concentration of 14 mg/mL to 18 mg/mL, or at a concentration of 14 mg/mL to 16 mg/mL. Specifically, the amino acid may be at a concentration of 15 mg/mL.

In an embodiment, the amino acid may be arginine, and may be preferably arginine-HCl. Here, the arginine may be included at a concentration of 14 mg/mL to 16 mg/mL, and may be preferably included at a concentration of 15 mg/mL.

In addition, the pharmaceutical formulation may further comprise a sugar. The sugar may be any one selected from the group consisting of sucrose, sorbitol, glycerol, trehalose, and mannitol. Here, the sugar may be included at a concentration of 120 mg/mL to 180 mg/mL. In addition, the sugar may be at a concentration of 120 mg/mL to 170 mg/mL, at a concentration of 120 mg/mL to 160 mg/mL, at a concentration of 120 mg/mL to 155 mg/mL, at a concentration of 130 mg/mL to 170 mg/mL, at a concentration of 130 mg/mL to 160 mg/mL, at a concentration of 130 mg/mL to 155 mg/mL, at a concentration of 135 mg/mL to 170 mg/mL, at a concentration of 135 mg/mL to 160 mg/mL, at a concentration of 135 mg/mL to 155 mg/mL, at a concentration of 140 mg/mL to 170 mg/mL, at a concentration of 140 mg/mL to 160 mg/mL, at a concentration of 140 mg/mL to 155 mg/mL, at a concentration of 145 mg/mL to 170 mg/mL, at a concentration of 145 mg/mL to 160 mg/mL, or at a concentration of 145 mg/mL to 155 mg/mL.

In an embodiment, the sugar may be sucrose, and the sucrose may be at a concentration of 150 mg/mL.

In an embodiment of the present invention, the pharmaceutical formulation may comprise (i) a fusion protein dimer comprising an IL-2 protein and a CD80 protein at a concentration of 3.0 mg/mL to 5.0 mg/mL; (ii) histidine at a concentration of 10 mM to 30 mM; (iii) poloxamer 188 at a concentration of 0.065 w/w % to 0.075 w/w %; (iv) polysorbate 80 at a concentration of 0.09 w/w % to 0.11 w/w %; (v) arginine at a concentration of 10 mg/mL to 30 mg/mL; and (vi) sucrose at a concentration of 120 mg/mL to 180 mg/mL, wherein the pH of the pharmaceutical formulation may be from 6.5 to 7.5.

The pharmaceutical formulation may be stored in a container selected from the group consisting of a vial, a cartridge, a syringe, and an autoinjector.

In addition, the container in which the formulation is stored may be stored at room temperature, at 2° C. to 8° C., or at 25° C. to 40° C. until administered to a subject in need of treatment.

The subject may be a mammal such as human, dog, cow, horse, pig, sheep, goat, cat, mouse, rabbit, and rat, and may be preferably human.

The formulation may be administered by parenteral administration such as subcutaneous administration, intravenous administration, mucosal administration, intramuscular administration, or intraperitoneal administration, but not limited thereto. Preferably, it may be intravenously administered.

### MODE FOR CARRYING OUT THE INVENTION

Hereinafter, the present invention will be described in more detail by way of the following examples. However, the following examples are only for illustrating the present invention, and the scope of the present invention is not limited thereto.

### Preparation Example 1. Preparation of hCD80-Fc-IL-2 Variant (2M): GI-101

In order to produce a fusion protein dimer comprising a human CD80 fragment, an Fc domain, and an IL-2 variant, a polynucleotide was synthesized through the Invitrogen GeneArt Gene Synthesis service of ThermoFisher Scientific. Specifically, the polynucleotide contains a nucleotide sequence (SEQ ID NO: 8) which encodes a fusion protein that contains a signal peptide (SEQ ID NO: 1), a CD80 fragment (SEQ ID NO: 2), an Ig hinge (SEQ ID NO: 3) to which a linker is bound, an Fc domain (SEQ ID NO: 4), a linker (SEQ ID NO: 5), and an IL-2 variant (2M) (R38A, F42A) (SEQ ID NO: 6) having two amino acid substitutions, in this order, from the N-terminus. The polynucleotide was inserted into pcDNA3_4 vector. In addition, the vector was introduced into CHO cells (Expi-CHO™) to express the fusion protein of SEQ ID NO: 9. After the vector was introduced, culture was performed for 7 days under the condition of 37° C., 125 RPM, and 8% CO₂. Then, the culture was collected and the fusion protein was purified therefrom. The purified fusion protein was designated “GI-101”.

Purification was carried out using chromatography containing MabSelect SuRe protein A resin. The fusion protein was bound thereto under a condition of 25 mM Tris, and 25 mM NaCl, and pH 7.4. Then, elution was performed with 100 mM NaCl and 100 mM acetic acid (pH 3). 20% 1 M Tris-HCl at pH 9 was placed in a collection tube, and then the fusion protein dimer was collected. For the collected fusion protein dimer, the buffer was exchanged through dialysis with PBS buffer for 16 hours.

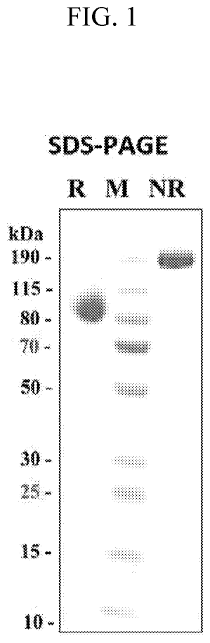

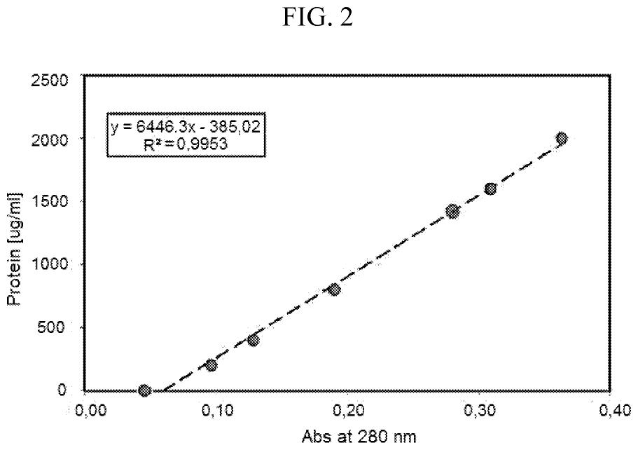

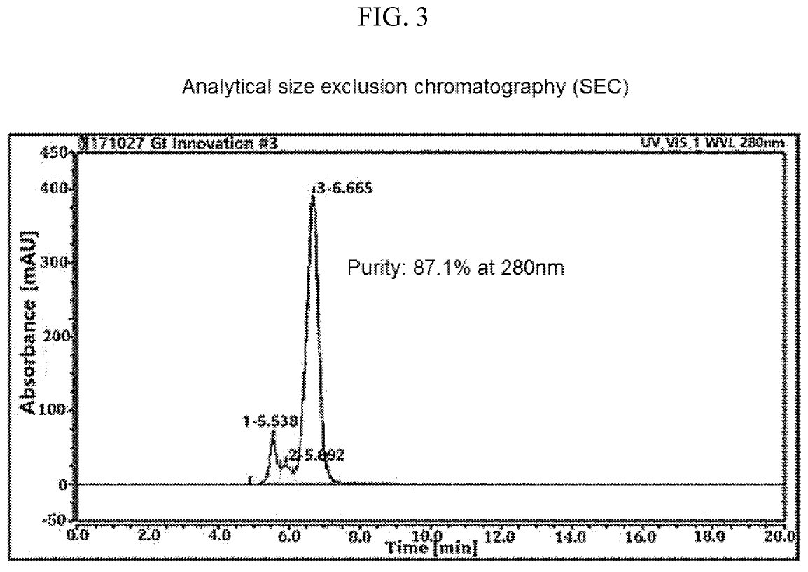

Thereafter, absorbance at 280 nm wavelength was measured, over time, with size exclusion chromatography using a TSKgel G3000SWXL column (TOSOH Bioscience), to obtain a highly concentrated fusion protein dimer. Here, the isolated and purified fusion protein dimer was subjected to SDS-PAGE under reduced (R) or non-reduced (NR) condition, and stained with Coomassie Blue to check its purity (FIG. 1). It was identified that the fusion protein dimer was contained at a concentration of 2.78 mg/ml when detected with NanoDrop (FIG. 2). In addition, the results obtained by analysis using size exclusion chromatography are provided in FIG. 3.

### Example 1. Evaluation of Optimal Buffer/pH Condition

In order to determine the optimal buffer/pH for the liquid formulation containing the fusion protein dimer (GI-101) of a CD80 protein and an IL-2 protein, a total of eight buffer/pH were screened. A stability test (40° C., 2 weeks) was performed to select the optimal buffer/pH.

The buffer/pH screening samples were stored at 40° C. for 2 weeks, and then the samples were analyzed using size exclusion chromatography (SEC). In order to identify the size exclusion profile of the GI-101 protein, SEC was performed using HPLC (Waters, e2695 and Thermo scientific, Ultimate 3000). The % area of the monomer was calculated using a chromatogram at 214 nm.

During formulation development, the SEC profile was used to aim at maximizing the % monomer. The stability test results for buffer/pH screening are summarized in Table 1. Sample #6 containing histidine buffer, pH 7.0 exhibited less change than the other samples.

TABLE 1

| | | SEC | Rate of change |
|---|---|---|---|
| No. | Buffer | (% monomer) | (%)* |
| 1 | citrate buffer, pH 4.5 | 0.00 | −100 |
| 2 | citrate buffer, pH 5.0 | 0.00 | −100 |
| 3 | histidine buffer, pH 5.5 | 0.00 | −100 |
| 4 | histidine buffer, pH 6.0 | 10.60 | −88.52 |
| 5 | histidine buffer, pH 6.5 | 6.48 | −92.95 |
| 6 | histidine buffer, pH 7.0 | 36.91 | −60.15 |
| 7 | phosphate buffer, pH 7.0 | 8.46 | −90.76 |
| 8 | phosphate buffer, pH 7.5 | 0.00 | −100 |

*Rate of change = (data after storage at 40° C. for 2 weeks − initial data)/initial data × 100

Based on the stability test data, a histidine buffer at pH 7.0 was determined as a buffer for GI-101.

TABLE 2

| Composition | Concentration |
|---|---|
| histidine buffer, pH 7.0 | 20 mM |

### Example 2. Excipient Screening Test

### Example 2.1. Excipient Screening

In order to determine the excipient for the liquid formulation containing GI-101, an excipient screening test was performed under the buffer/pH (histidine buffer, pH 7.0) condition selected in Example 1.

For 8 different excipients [Polysorbate 80, Poloxamer 188, arginine-HCl (L-Arginine Monohydrochloride), L-Methionine, D-Mannitol, Sorbitol, Sucrose, and D-(+)-Trehalose Dihydrate], the results of Tm & Tagg, SEC and visual particle test obtained by performing under 5 different test conditions were statistically analyzed to select poloxamer 188, arginine-HCl, and sucrose as excipients.

TABLE 3

| Type of excipient | Surfactant | Amino acid | Sugar |
|---|---|---|---|
| Selected | poloxamer 188 | arginine-HCl | sucrose |

### Example 2.2. Screening for Optimal Excipient Concentration

According to Example 2.1, poloxamer 188, arginine-HCl, and sucrose were selected as excipients for the liquid formulation containing GI-101.

Screening tests were performed under 16 conditions by varying the concentration of each excipient in order to find the optimal concentration for the combination of the three excipients.

### Example 2.2.1. Thermal Stability Test (40° C., 2 Weeks)

Sixteen excipient screening test samples were stored at 40° C. for 2 weeks, and then the samples were analyzed through the protein concentration (A280) and SEC tests. The results are shown in Table 4.

TABLE 4

| | A280 | | | |
|---|---|---|---|---|
| | Protein | | SEC | |
| | concentration | Rate of change | Monomer | Rate of |
| No. | (mg/mL) | (%) | (%) | change (%) |
| 1 | 8.38 | 1.3% | 42.86 | −54.4% |
| 2 | 8.45 | −0.2% | 67.26 | −28.3% |
| 3 | 8.69 | 4.8% | 14.42 | −84.6% |
| 4 | 8.37 | 0.2% | 63.08 | −32.6% |
| 5 | 8.33 | −1.9% | 63.37 | −32.5% |
| 6 | 8.45 | 1.7% | 42.86 | −53.9% |
| 7 | 8.74 | 4.8% | 14.97 | −84.1% |
| 8 | 8.46 | 1.2% | 38.19 | −59.2% |
| 9 | 8.46 | 1.3% | 64.98 | −30.6% |
| 10 | 8.35 | −1.1% | 81.92 | −12.5% |
| 11 | 8.44 | 0.2% | 81.28 | −12.8% |
| 12 | 8.45 | 0.5% | 61.95 | −33.6% |
| 13 | 8.49 | 0.1% | 65.6 | −30.0% |
| 14 | 8.43 | 0.8% | 65.26 | −30.3% |
| 15 | 8.45 | 1.4% | 39.29 | −58.1% |
| 16 | 8.41 | 0.5% | 80.12 | −14.4% |

### Example 2.2.2. RSM and Simulation Results

RSM (Response surface model) was used to determine the optimal concentration of each excipient. The optimal reaction could be found by adjusting the concentration of each excipient in the prediction profiler. In the results predicted by the prediction profiler, the concentration of sucrose was set to be the optimal value of 150 mg/mL, which is the maximum value of the experiment. Therefore, only arginine-HCl and poloxamer 188 concentrations were set by simulation. The predicted optimal concentration range of each excipient is shown in Table 5.

TABLE 5

| Excipient | Range | |
|---|---|---|
| poloxamer 188 | 0.05 to 0.075 | w/w % |
| arginine-HCl | 10 to 20 | mg/mL |
| sucrose | 150 | mg/mL |

### Example 2.3. Final Stability Test (4 Weeks)

Based on the optimal concentration range for each excipient in Table 5, the final formulation candidates in Table 6 were derived. The final 4 weeks stability test was performed on the formulation candidates in Table 6.

TABLE 6

| Excipient | Poloxamer 188 | | Arginine-HCl | | Sucrose | |
|---|---|---|---|---|---|---|
| Optimal | 0.07 | w/w % | 15 | mg/mL | 150 | mg/mL |
| condition | | | | | | |
| Target range | 0.065 to 0.075 | w/w % | 14 to 16 | mg/mL | 140 to 160 | mg/mL |

The stability of the formulation candidates was tested under a total of five different conditions. Immediately after preparing samples, the release test (t=0) was performed, and candidate samples were stored for 4 weeks at 5° C. (long-term condition), −70° C. (long-term second condition), 25C (accelerated condition), and 40° C. (severe condition), respectively. In particular, in the case of the 40° C. stability test (severe condition), an additional sample was taken at 2-week time point in order to identify the change trend. After storing for 4 weeks under each condition, the samples were analyzed though SEC, protein concentration (A280) and pH tests. The final stability test results for the GI-101 formulation candidate are shown in Table 7.

TABLE 7

| | Protein | | |
|---|---|---|---|
| | concentration | | SEC |
| Test condition | (mg/mL) | pH | (% monomer) |
| Long-term condition | 8.01 | 7.11 | 94.50 |
| (5° C., 4 weeks) | | | |
| Long-term second condition | 8.01 | 7.08 | 94.30 |
| (−70° C., 4 weeks) | | | |
| Accelerated condition | 8.05 | 7.11 | 94.90 |
| (25° C., 4 weeks) | | | |
| Severe condition | 8.15 | 7.15 | 78.13 |
| (40° C., 2 weeks) | | | |
| Severe condition | 8.12 | 7.05 | 65.00 |
| (40° C., 4 weeks) | | | |

As a result of the stability test, the formulation candidate exhibited a stable state under the condition of −70° C., 5° C., and 25° C. From this result, the final formulation for GI-101 was determined to be 8 mg/mL of GI-101, 20 mM of histidine buffer (pH 7.0), 0.07 w/w % of poloxamer 188, 15 mg/mL of arginine-HCl and 150 mg/mL of sucrose.

TABLE 10

| Substance | Target concentration | | Range | |
|---|---|---|---|---|
| GI-101 | 8 | mg/mL | 37.2 to 8.8 | mg/mL |
| Buffer | histidine buffer: 20 | mM | N/A | |
| pH | pH | 7.0 | pH | 6.8 to 7.2 |
| Surfactant | poloxamer 188: 0.07 | w/w % | 0.065 to 0.075 | w/w % |
| Amino acid | arginine-HCl: 15 | mg/mL | 14 to 16 | mg/mL |
| Sugar | sucrose: 150 | mg/mL | 140 to 160 | mg/mL |

### Example 3. Polysorbate 80 Addition Test

In the process of producing the GI-101 raw material medicine using the formulation candidate determined in Example 2.3, visible particles were found, and a formulation was additionally developed. It was identified that visible particles appear when silicone present in the raw material medicine binds to protein by physical stress to form a silicone-protein complex. Since there is a limit to removing silicone or alleviating physical stress in the process of producing the raw material medicine, a new formulation that inhibits the formation of a silicone-protein complex was developed to solve the problem. It has been reported that poloxamer 188 is an inefficient surfactant for inhibiting the formation of a silicone-protein complex, and based on this, an experiment was performed to add polysorbate 80 (PS80) as a surfactant.

Specifically, the test was performed by adding polysorbate 80 by concentrations (0 w/w %, 0.02 w/w %, 0.04 w/w %, 0.06 w/w %, 0.08 w/w %, and 0.1 w/w %) to GI-101 DS (4 mg/mL of GI-101, 20 mM of histidine buffer (pH 7.0), 0.07 w/w % of poloxamer 188, 15 mg/mL of arginine-HCl, and 150 mg/mL of sucrose). The concentration of GI-101 was adjusted to 4 mg/mL in order to increase long-term stability.

### Example 3.1. Visual Particle Observation

Each of the three tubes was tested for each condition, and even when particles were observed only in one of the three tubes, it was labeled as O (i.e., ‘particle observation’) (Table 9).

TABLE 9

| | | GI-101 DS + | GI-101 DS + | GI-101 DS + | GI-101 DS + | GI-101 DS + | GI-101 DS + | 8 mg/mL |
|---|---|---|---|---|---|---|---|---|
| | | 0 w/w % | 0.02 w/w % | 0.04 w/w % | 0.06 w/w % | 0.08 w/w % | 0.1 w/w % | GI-101 |
| Condition | | PS80 | PS80 | PS80 | PS80 | PS80 | PS80 | w/o PS80 |
| Release time | | X | X | X | X | X | X | X |
| point (0 Time) | | | | | | | | |
| Physical stress | | ◯ | not | not | not | not | not | ◯ |
| (continuous) | | | tested | tested | tested | tested | tested | |
| Week 1 | physical | X | X | X | X | X | X | not |
| | stress | | | | | | | tested |
| | storage at | X | X | X | X | X | X | not |
| | 5° C. | | | | | | | tested |
| | storage at | X | X | X | X | X | X | not |
| | 25° C. | | | | | | | tested |
| Week 2 | physical | X | X | X | X | X | X | not |
| | stress | | | | | | | tested |
| | storage at | X | X | X | X | X | X | not |
| | 5° C. | | | | | | | tested |
| | storage at | X | X | X | X | X | X | not |
| | 25° C. | | | | | | | tested |
| Week 3 | physical | ◯ | X | ◯ | X | X | X | not |
| | stress | | | | | | | tested |
| | storage at | ◯ | X | ◯ | X | X | X | not |
| | 5° C. | | | | | | | tested |
| | storage at | ◯ | ◯ | ◯ | ◯ | X | X | not |
| | 25° C. | | | | | | | tested |
| Week 4 | physical | not | not | not | not | X | X | not |
| | stress | tested | tested | tested | tested | | | tested |
| | storage at | not | not | not | not | X | X | not |
| | 5° C. | tested | tested | tested | tested | | | tested |
| | storage at | not | not | not | not | X | X | not |
| | 25° C. | tested | tested | tested | tested | | | tested |

In the samples to which 0.08 w/w % or more of PS80 was added, no particles were observed until 4 weeks. In the case of a sample in which particles were observed at Week 3, the experiment at Week 4 was not performed.

### Example 3.2. Evaluation of Quality Effect

In order to investigate the quality effect of the addition of polysorbate 80 (PS80), the protein concentration, charge variation, and purity of the sample to which 0.1 w/w % PS80 was added were measured. As a result of observing the sample to which 0.1 w/w % PS80 was added for up to 4 weeks, it was identified that the quality was maintained. The measurement results for each condition are shown in Table 10.

TABLE 10

| | | | | GI-101 DS + | GI-101 DS + | GI-101 DS + |
|---|---|---|---|---|---|---|
| | | | GI-101 DS + | 0.1 w/w % | 0.1 w/w % | 0.1 w/w % |
| | | | 0.1 w/w % | PS80 | PS80 | PS80 |
| | | | PS80 | physical | 5° C., | 25° C., |
| Analysis tool | | Unit | 0T | stress | 4 weeks | 4 weeks |
| A280 (protein amount) | | mg/mL | 4.23 | 4.22 | 4.21 | 4.23 |
| icIEF | Peak 1- | Area % | 11.88 (pI: 5.32) | 11.21 (pI: 5.31) | 11.19 (pI: 5.32) | 12.11 (pI: 5.32) |
| | Information | | | | | |
| | only | | | | | |
| | Peak 2- | Area % | 23.99 (pI: 5.48) | 25.21 (pI: 5.50) | 24.53 (pI: 5.50) | 26.77 (pI: 5.48) |
| | Information | | | | | |
| | only | | | | | |
| | Peak 3- | Area % | 64.13 (pI: 5.62) | 63.58 (pI: 5.63) | 64.28 (pI: 5.60) | 61.13 (pI: 5.60) |
| | Information | | | | | |
| | only | | | | | |
| SE- | HMWS | % | 1.43 | 1.66 | 1.63 | 1.39 |
| HPLC | Monomer | % | 98.2 | 97.96 | 97.97 | 97.9 |
| | LMWS | % | 0.37 | 0.38 | 0.4 | 0.71 |

Considering that the addition of PS80 at a high concentration is effective in reducing visible particles, the concentration of PS80 was determined to be 0.1 w/w %. By combining these results, the final formulation composition for GI-101 was determined as shown in Table 11 below.

TABLE 11

| Substance | Target concentration | | Range | |
|---|---|---|---|---|
| GI-101 | 4 | mg/mL | 3.6 to 4.4 | mg/mL |
| Buffer | histidine buffer: 20 | mM | N/A | |
| pH | pH | 7.0 | pH | 6.8 to 7.2 |
| Surfactant | poloxamer 188: 0.07 | w/w % | 0.065 to 0.075 | w/w % |
| | polysorbate 80: 0.1 | w/w % | 0.09 to 0.11 | w/w % |
| Amino acid | arginine-HCl: 15 | mg/mL | 14 to 16 | mg/mL |
| Sugar | sucrose: 150 | mg/mL | 140 to 160 | mg/mL |

1. A pharmaceutical formulation comprising:
(i) a fusion protein dimer comprising an IL-2 protein and a CD80 protein at a concentration of 3.0 mg/mL to 5.0 mg/mL;
(ii) a buffer at a concentration of 10 mM to 30 mM; and
(iii) a surfactant at a concentration of 0.155 w/w % to 0.185 w/w %;
wherein, the pH of the formulation is from 6.5 to 7.5,
wherein a fusion protein consists of the following structural formula (I) or (II): 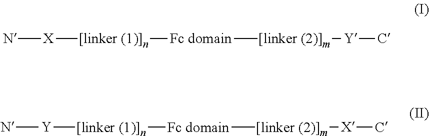
Here, in the structural formulas (I) and (II),
N′ is the N-terminus of the fusion protein,
C′ is the C-terminus of the fusion protein,
X is a CD80 protein,
Y is an IL-2 protein,
the linkers (1) and (2) are peptide linkers, and
n and m are each independently 0 or 1.

2. The pharmaceutical formulation of claim 1, wherein the fusion protein dimer is at a concentration of 3.6 mg/mL to 4.4 mg/mL.

3. The pharmaceutical formulation of claim 1, wherein the buffer is histidine.

4. The pharmaceutical formulation of claim 3, wherein the histidine is at a concentration of 20 mM.

5. The pharmaceutical formulation of claim 1, wherein the pH of formulation is from 6.8 to 7.2.

6. The pharmaceutical formulation of claim 5, wherein the pH of the formulation is 7.0.

7. The pharmaceutical formulation of claim 1, wherein the surfactant is two surfactants selected from the group consisting of polysorbate 20, polysorbate 28, polysorbate 40, polysorbate 60, polysorbate 65, polysorbate 80, polysorbate 81, polysorbate 85, poloxamer 181, poloxamer 188, poloxamer 407 and polyethylene glycol (PEG).

8. The pharmaceutical formulation of claim 7, wherein the two surfactants are poloxamer 188 and polysorbate 80.

9. The pharmaceutical formulation of claim 8,
wherein the poloxamer 188 is at a concentration of 0.065 w/w % to 0.075 w/w %, and
the polysorbate 80 is at a concentration of 0.09 w/w % to 0.11 w/w %.

10. The pharmaceutical formulation of claim 1, wherein the pharmaceutical formulation further comprises an amino acid.

11. The pharmaceutical formulation of claim 10, wherein the amino acid is any one selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan.

12. The pharmaceutical formulation of claim 11, wherein the amino acid is arginine.

13. The pharmaceutical formulation of claim 10, wherein the amino acid is at a concentration of 10 mg/mL to 30 mg/mL.

14. The pharmaceutical formulation of claim 13, wherein the amino acid is at a concentration of 14 mg/mL to 16 mg/mL.

15. The pharmaceutical formulation of claim 14, wherein the amino acid is at a concentration of 15 mg/mL.

16. The pharmaceutical formulation of claim 1, wherein the pharmaceutical formulation further comprises a sugar.

17. The pharmaceutical formulation of claim 16, wherein the sugar is any one selected from the group consisting of sucrose, sorbitol, glycerol, trehalose and mannitol.

18. The pharmaceutical formulation of claim 16, wherein the sugar is at a concentration of 120 mg/mL to 180 mg/mL.

19. The pharmaceutical formulation of claim 18, wherein the sugar is at a concentration of 140 mg/mL to 160 mg/mL.

20. The pharmaceutical formulation of claim 17, wherein the sugar is sucrose.

21. The pharmaceutical formulation of claim 20, wherein the sucrose is at a concentration of 150 mg/mL.

22. A pharmaceutical formulation comprising:
(i) a fusion protein dimer comprising an IL-2 protein and a CD80 protein at a concentration of 3.0 mg/mL to 5.0 mg/mL;
(ii) histidine at a concentration of 10 mM to 30 mM;
(iii) poloxamer 188 at a concentration of 0.065 w/w % to 0.075 w/w %;
(iv) polysorbate 80 at a concentration of 0.09 w/w % to 0.11 w/w %;
(v) arginine at a concentration of 10 mg/mL to 30 mg/mL; and
(vi) sucrose at a concentration of 120 mg/mL to 180 mg/mL,
wherein, the pH of the formulation is from 6.5 to 7.5,
wherein a fusion protein consists of the following structural formula (I) or (II): 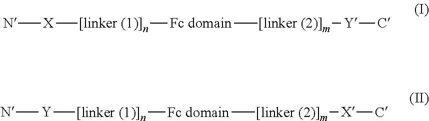
Here, in the structural formulas (I) and (II),
N′ is the N-terminus of the fusion protein,
C′ is the C-terminus of the fusion protein,
X is a CD80 protein,
Y is an IL-2 protein,
the linkers (1) and (2) are peptide linkers, and
n and m are each independently 0 or 1.

23. The pharmaceutical formulation of claim 22, wherein the formulation is an intravenous formulation.

24. The pharmaceutical formulation of claim 22, wherein the formulation is for the prevention or treatment of cancer or an infectious disease.
